(19) United States
(12) Patent Application Publication
(10) Pub. No.: US 2021/0353820 A1
(43) Pub. Date: Nov. 18, 2021

(54) FAN SYSTEM FOR IMPROVING INDOOR AIR QUALITY AND RELATED METHODS

(71) Applicant: DELTA T, LLC, Lexington, KY (US)

(72) Inventor: Thomas Lesser, Chicago, IL (US)

(21) Appl. No.: 17/320,829

(22) Filed: May 14, 2021

Related U.S. Application Data
(60) Provisional application No. 63/025,501, filed on May 15, 2020.
(60) Provisional application No. 63/029,105, filed on May 22, 2020.
(60) Provisional application No. 63/038,446, filed on Jun. 12, 2020.
(60) Provisional application No. 63/045,882, filed on Jun. 30, 2020.
(60) Provisional application No. 63/060,826, filed on Aug. 4, 2020.

(51) Int. Cl. A61L 9/20; F24F 7/007; F24F 8/22

(52) U.S. Cl. CPC A61L 9/205; F24F 7/007; A61L 2209/12; A61L 2209/111; F24F 8/22

(57) ABSTRACT

A system for minimizing exposure of at least one occupant to a pathogen and for circulating air within a space is provided. The system includes a fan for circulating air within the space. At least one sensor for sensing a parameter within the space is provided. A controller is configured for determining a risk of exposure to the pathogen based on the sensed parameter within the space and to alter operation of the fan and/or a source of germicidal energy, such as a UV light, based on the determined risk of exposure. Related methods are also disclosed.

This application claims the benefit of U.S. Provisional Patent Application No. 63/025,501, filed May 15, 2020, U.S. Provisional Patent Application Ser. No. 63/029,105, filed May 22, 2020, U.S. Provisional Patent Application Ser. No. 63/038,446, filed Jun. 12, 2020, U.S. Provisional Patent Application Ser. No. 63/045,882, filed Jun. 30, 2020, and U.S. Provisional Patent Application Ser. No. 63/060,826, filed Aug. 4, 2020, the disclosures of which are incorporated herein by reference.

### TECHNICAL FIELD

This application relates to the air circulation arts and, more particularly, to a fan adapted for improving indoor air quality, such as by minimizing the presence of airborne pathogens, and related methods.

### BACKGROUND

A variety of fan systems have been made and used over the years in a variety of contexts. For instance, various ceiling fans are disclosed in U.S. Pat. No. 7,284,960, entitled “Fan Blades,” issued Oct. 23, 2007; U.S. Pat. No. 6,244,821, entitled “Low Speed Cooling Fan,” issued Jun. 12, 2001; U.S. Pat. No. 6,939,108, entitled “Cooling Fan with Reinforced Blade,” issued Sep. 6, 2005; and U.S. Pat. No. D607,988, entitled “Ceiling Fan,” issued Jan. 12, 2010. The disclosures of each of those U.S. patents are incorporated by reference herein. Additional exemplary fans are disclosed in U.S. Pat. Pub. No. 2008/0008596, entitled “Fan Blades,” published Jan. 10, 2008; U.S. Pat. Pub. No. 2009/0208333, entitled “Ceiling Fan System with Brushless Motor,” published Aug. 20, 2009; and U.S. Pat. Pub. No. 2010/0278637, entitled “Ceiling Fan with Variable Blade Pitch and Variable Speed Control,” published Nov. 4, 2010, the disclosures of which are also incorporated by reference herein. It should be understood that teachings herein may be incorporated into any of the fans described in any of the above-referenced patents, publications, or patent applications. It should also be understood that a fan may include sensors or other features that are used to control, at least in part, operation of a fan system. For instance, such fan systems are disclosed in U.S. Pat. Pub. No. 2009/0097975, entitled “Ceiling Fan with Concentric Stationary Tube and Power-Down Features,” published Apr. 16, 2009, the disclosure of which is incorporated by reference herein; U.S. Pat. Pub. No. 2009/0162197, entitled “Automatic Control System and Method to Minimize Oscillation in Ceiling Fans,” published Jun. 25, 2009, the disclosure of which is incorporated by reference herein; U.S. Pat. Pub. No. 2010/0291858, entitled “Automatic Control System for Ceiling Fan Based on Temperature Differentials,” published Nov. 18, 2010, the disclosure of which is incorporated by reference herein; and U.S. Provisional Patent App. No. 61/165,582, entitled “Fan with Impact Avoidance System Using Infrared,” filed Apr. 1, 2009, the disclosure of which is incorporated by reference herein. Alternatively, any other suitable control systems/features may be used in conjunction with embodiments described herein.

In some environments, it is desirable to sterilize the air and/or remove airborne diseases and disease vectors from the air. Existing methods for reducing airborne disease transmission between room occupants include fresh air ventilation, filtration, and direct deactivation/destruction methods such as irradiation or oxidation of the pathogens themselves. For instance, this can be achieved through the use of ultraviolet radiation (which is often referred to as ultraviolet germicidal irradiation (UVGI)). UVGI is a disinfection method that uses ultraviolet (UV) light at sufficiently short wavelength to kill living microorganisms. It is used in a variety of applications, such as food, air and water purification. UVGI utilizes short-wavelength ultraviolet radiation (UV-C) that is harmful to microorganisms. It is effective in destroying the nucleic acids in these organisms so that their DNA is disrupted by the UV radiation, leaving them unable to perform vital cellular functions.

As can be appreciated, a germicidal or UV fixture positioned in a space is somewhat effective, but obviously limited in efficacy given its stationary nature (and the use of multiple stationary devices may be considered costly and inefficient in most applications). In an effort to address this shortcoming, others have in the past proposed connecting UV devices to ceiling fans, but significant limitations remain. For instance, U.S. Pat. No. 4,422,824 proposed incorporating a light into a fan blade, which obviously may impact the appearance and aerodynamic features of the blade, and otherwise makes for a complicated arrangement. More recently, U.S. Pat. No. 7,897,299 proposed a housing for containing the UV lighting separate from the fan, but this configuration suffers from the disadvantage that the light projects downward onto the fan blades and may create an annoying strobe effect. The housing also restricts the air flow within the field of the light, and thus is of limited effect, at best.

While fundamentally effective, many of these approaches are not implemented successfully due to lack of operator training, maintenance issues, sub-par user interfaces and experiences, and cost. Accordingly, a need is identified for an improved manner of providing a fan with a sterilizing capability and, in particular, a degree of automation of operating certain sterilizing functions, whether as part of the fan or not, that avoids the problems associated with the above-mentioned approaches.

### SUMMARY

According to one aspect of the disclosure, an apparatus for sterilizing and circulating air in a space is provided. The apparatus comprises a fan, a source of germicidal energy, a sensor for sensing a parameter of the space, and a controller for controlling the fan or the source based on the sensed parameter.

In one embodiment, the source comprises one or more UV lights attached to the fan. A reflector may serve to reflecting light from the source onto a photocatalyst. The reflector may substantially surround the light source, and may comprise a plurality of substantially parallel plates, each having a reflective surface. The controller may be adapted to control the source based on the sensed parameter.

According to another aspect of the disclosure, a system for minimizing exposure of at least one occupant to a pathogen and for circulating air within a space is provided. The system includes a fan, at least one sensor for sensing a parameter within the space, and a controller configured for determining a risk of exposure to the pathogen based on the sensed parameter within the space and to alter operation of the fan based on the determined risk of exposure.

In one embodiment, the at least one sensor comprises at least one of a temperature sensor, a humidity sensor, an occupancy sensor, and a carbon dioxide sensor. The controller may be configured to alter a speed of the fan in response to the determined risk of exposure. The fan may further include a generator of germicidal energy, such as a UV light, and the controller may be configured to activate the light based on the determined risk of exposure. The determined risk of exposure may be based at least partially on a measured level of carbon dioxide in the space.

According to a further aspect of the disclosure, a method for circulating air within a space is provided. The method comprises controlling a fan for circulating air within the space based on a determined risk of exposure to a pathogen. The method may further include the steps of sensing a parameter within the space, and determining the risk of exposure. The step of determining the risk of exposure may comprise determining a CO2 concentration or CO2 generation rate within the space, or determining the risk of exposure comprises determining a probability of exposure.

The method may further include the step of controlling a source of germicidal energy based on a determined risk of exposure to a pathogen. Still further, the method may include the step of controlling a source of UV light associated with the space based on a determined risk of exposure to a pathogen.

### BRIEF DESCRIPTION OF THE DRAWINGS

The accompanying drawing figures incorporated herein and forming a part of the specification, illustrate several aspects of the disclosure and, together with the description, serve to explain certain principles thereof. In the drawing figures:

FIG. 1 is a partially schematic perspective view illustrating a system for minimizing the risk of an occupant of being exposed to airborne pathogens; and

FIGS. 2-5 illustrate various embodiments of ceiling fans for possible inclusion in the system of FIG. 1.

The following description of certain examples of the invention should not be used to limit the scope of the present disclosure. Other examples, features, aspects, embodiments, and advantages of the invention will become apparent to those skilled in the art from the following description, which includes by way of illustration, one or more of the best modes contemplated for carrying out the invention. As will be realized, the invention is capable of other different and obvious aspects, all without departing from the invention. Accordingly, the drawings and descriptions should be regarded as illustrative in nature and not restrictive.

### DETAILED DESCRIPTION

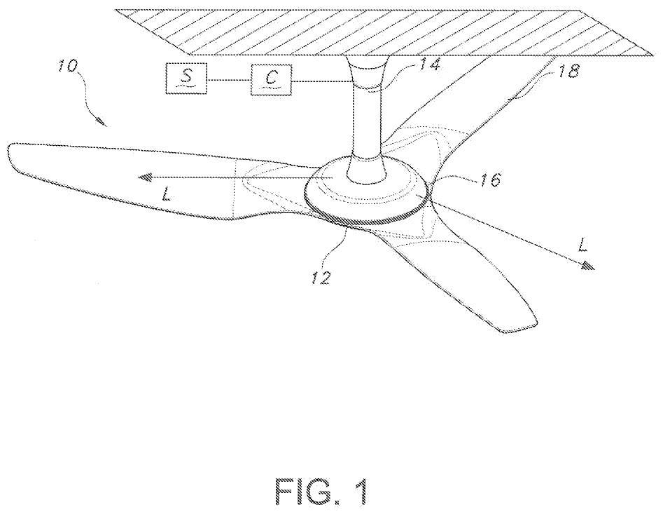

Referring to FIG. 1, an exemplary fan 10 according to one embodiment comprises a hub 12, which may include a motor (which may be enclosed or contained within or adjacent to hub 12). The fan 10 may be connected to a support 14, and may also include a plurality of fan blades 18. In the present example, fan 10 (including hub 12 and fan blades 18) has a diameter of approximately 2-8 feet. In other variations, fan 10 has a diameter of up to 24 feet. Alternatively, fan 10 may have any other suitable dimensions depending on a particular application.

Support 14 is configured to be coupled to a surface (such as a ceiling) or other stable support structure (such as a joist, beam, or the like) at a first end such that fan 10 is substantially attached to the surface or other structure. Support 14 of the present example comprises an elongate tube-like structure that couples fan 10 to a ceiling, though it should be understood that support 14 may be constructed and/or configured in a variety of other suitable ways as will be apparent to one of ordinary skill in the art in view of the teachings herein. By way of example only, support 14 need not be coupled to a ceiling or other overhead structure, and instead may be coupled to a wall or to the ground. For instance, support 14 may be positioned on the top of a post that extends upwardly from the ground. Alternatively, support 14 may be mounted in any other suitable fashion at any other suitable location. By way of example only, support 14 may be configured in accordance with the teachings of U.S. Pat. Pub. No. 2009/0072108, entitled “Ceiling Fan with Angled Mounting,” published Mar. 19, 2009, the disclosure of which is incorporated by reference herein. As yet another alternative, support 14 may have any other suitable configuration. Furthermore, support 14 may be supplemented in numerous ways. One merely illustrative example is described in detail below, while other examples and variations will be apparent to those of ordinary skill in the art in view of the teachings herein.

The motor may comprise an AC induction motor having a drive shaft, though it should be understood that motor may alternatively comprise any other suitable type of motor (e.g., a permanent magnet brushless DC motor, a brushed motor, an inside-out motor, etc.). In the present example, motor is fixedly coupled to support 14 and rotatably coupled to hub 12. Furthermore, motor is operable to rotate hub 12 and the plurality of fan blades 18. By way of example only, motor may be constructed in accordance with at least some of the teachings of U.S. Pat. Pub. No. 2009/0208333, entitled “Ceiling Fan System with Brushless Motor,” published Aug. 20, 2009, the disclosure of which is incorporated by reference herein. Furthermore, fan 10 may include control electronics that are configured in accordance with at least some of the teachings of U.S. Pat. Pub. No. 2010/0278637, entitled “Ceiling Fan with Variable Blade Pitch and Variable Speed Control,” published Nov. 4, 2010, the disclosure of which is incorporated by reference herein. Alternatively, motor may have any other suitable components, configurations, functionalities, and operability, as will be apparent to those of ordinary skill in the art in view of the teachings herein.

Hub 12 may be constructed in accordance with at least some of the teachings of U.S. Pat. Pub. No. 2010/0278637, entitled “Ceiling Fan with Variable Blade Pitch and Variable Speed Control,” published Nov. 4, 2010, the disclosure of which is incorporated by reference herein. Alternatively, hub 12 may be constructed in accordance with any of the teachings or other patent references cited herein. Still other suitable ways in which hub 12 may be constructed will be apparent to those of ordinary skill in the art in view of the teachings herein. It should also be understood that an interface component (not shown) may be provided at the interface of each fan blade 18 and hub 12. By way of example only, such an interface component may be configured in accordance with the teachings of U.S. Pat. Pub. No. 2009/0081045, entitled “Aerodynamic Interface Component for Fan Blade,” published Mar. 26, 2009, the disclosure of which is incorporated by reference herein. Of course, such an interface component may be omitted if desired.

Fan blades 18 may further be constructed in accordance with some or all of the teachings of any of the patents, patent publications, or patent applications cited herein. For example, fan blades 18 may be configured in accordance with the teachings of U.S. Pat. No. 7,284,960, entitled “Fan Blades,” issued Oct. 23, 2007; U.S. Pat. No. 6,244,821, entitled “Low Speed Cooling Fan,” issued Jun. 12, 2001; and/or U.S. Pat. No. 6,939,108, entitled “Cooling Fan with Reinforced Blade,” issued Sep. 6, 2005. The disclosures of each of those U.S. patents are incorporated by reference herein. As another merely illustrative example, fan blades 18 may be configured in accordance with the teachings of U.S. Pat. Pub. No. 2008/0008596, entitled “Fan Blades,” published Jan. 10, 2008, the disclosure of which is also incorporated by reference herein. As yet another merely illustrative example, fan blades 18 may be configured in accordance with the teachings of U.S. Pat. Pub. No. 2010/0104461, entitled “Multi-Part Modular Airfoil Section and Method of Attachment Between Parts,” published Apr. 29, 2010, the disclosure of which is incorporated by reference herein. Alternatively, any other suitable configurations for fan blades 18 may be used in conjunction with the examples described herein. For example, fan blades 18 may be formed of aluminum through an extrusion process such that each fan blade has a substantially uniform cross section along its length. It should be understood that fan blades 18 may alternatively be formed using any suitable material, or combination of materials, by using any suitable technique, or combination of techniques, and may have any suitable cross-sectional properties or other properties as will be apparent to one of ordinary skill in the art in view of the teachings herein.

According to one aspect, this disclosure relates to an air circulation device, such as fan 10, that may form part of a system that calculates a risk of transmission of one or more pathogens, diseases or disease vectors (collectively referred to as “pathogens”) at any given time and implements changes to address the presence of these pathogens. For instance, the fan may be associated with or include integral germ-killing technologies, such as photocatalytic oxidation, UVGI lighting, ion generators, or the like.

In assessing risk associated with various pathogens, the system may cause the fan to adjust speed and/or direction, may turn a germicidal generator (e.g., one or more UV lights or an ion generator, which may be attached to or separate from the fan) on/off, may turn photocatalytic lights on/off, may turn ventilation fans on/off, and/or may adjust ventilation fan(s) airflow rate. The fan 10 may provide operators with maintenance notifications based on real-time performance feedback. Wireless controls, strategies, and user interfaces may be employed, which may drastically reduce installation costs.

The fan 10 may include one or more sensors S for detecting environmental parameters within a given space that are indicative of indoor air quality. For instance, the fan 10 may include, be associated with, and/or be in communication with sensors for sensing temperature, humidity, occupancy, sound, light, and/or carbon dioxide level. The sensors S may be connected directly to the fan 10, or may form part of a separate structure within the room (e.g., wall controller) that may communicate with and provide signals for controlling the fan (such as by wireless communication).

Various manners exist for determining a risk of an airborne disease being transmitted to another person based on measured environmental parameters, such as, for example, risk of airborne disease transmission as a function of “rebreathed” air in a space, which correlates to the carbon dioxide level. According to one manner of determining a risk of airborne disease transmission, the probability P of exposure may be evaluated as follows:

$$D / S = [ 1 - exp ( - \frac{\overline{f} Iqt}{n} ) ]$$

Another measure involves determining a ventilation rate based on a function of indoor CO₂ concentration, which may be expressed by:

$$Q = \frac{G}{{C}_{in} - {C}_{out}}$$

The critical rebreathed fraction (f̅ₑ) represents the fraction of ambient CO₂ under which a reduction in transmission would be expected to occur. Substituting (f̅ₑ) for the indoor CO₂ concentration yields:

$$Q = \frac{G}{\overline{{f}_{c}} - {C}_{out}}$$

The indoor CO₂ generation rate may be expressed by

G=V̅Cₑₓ

These equations take one or more of the following parameters into consideration:

| Parameter | Description | Range |
|---|---|---|
| D | Number of infections (effective contact rate | (1-30) |
| | goal for TB control) | |
| S | Number of susceptibles (n-1) | (1-120) |
| f̅ | Average fraction of indoor air that is exhaled | — |
| f̅_{c} | Critical rebreathed fraction for indoor CO₂ | — |
| | concentration | |
| I | Number of infectors | 1-2 |
| q | Quantum generation rate | (1-13) |
| t | Total exposure time | (1-900) |
| n | Persons in a given space | (2-121) |
| Q | Ventilation rate (l/s per person) | — |
| G | Indoor CO₂ generation rate (l/s per person) | 3800-6460 |
| Cᵢₙ | Indoor CO₂ concentration (ppm) | (400-6000) |
| Cₒᵤₜ | Outdoor CO₂ concentration | — |
| V̅ | Average volume of gas exhaled for | 0.1-0.17 |
| | adolescents | |
| Cₑₓ | CO₂ concentration in exhaled breath | — |

Environmental factors that may affect the risk of pathogens and/or pathogen transmission within a space include occupancy, duration of occupancy, ventilation flow rate, CO₂ concentration and generation rate. In addition, factors such as air temperature and relative humidity may be important in addressing pathogens in the space, since these factors may impact UV effectiveness. Ventilating a room prior to a space being occupied, rather than waiting for risk to build, may be accomplished based on a time of day and a known time of occupancy of the space. Assessments of these factors can be used to preempt risk by making changes when known occupancies will occur. For instance, the following represents actions that may be taken based upon an assessed probability:

| | Low risk | High risk |
|---|---|---|
| P (probability, which | 0-5% | 20-90% |
| also equals D/S) | | |
| Indoor CO₂ concentration | 300-1000 pm | 1500 ppm+ |
| Fan speed | slow | medium |
| UV energy output | low | high |
| Photocatalyic activity | low | high |
| Outdoor air ventilation | off | on |

Sample calculations

| | low risk | high risk |
|---|---|---|
| | scenario | scenario |
| D | 1 | 20 |
| S | 120 | 100 |
| d/s (=P) | 0.83% | 20.00% |

As can be appreciated, the above presents an example only, and the probability values may vary depending on a particular situation or application.

According to one aspect of the disclosure, the fan 10 may form part of a system and thereby utilize output from the one or more sensors S associated therewith in order to operate based on a potential risk of exposure to air pathogens. A corresponding controller C may implement the output from the one or more sensors S into one or more of the equation(s) above for the purpose of enacting real time responses based on the conditions in a given space. For instance, the controller C may use output from the occupancy or carbon dioxide sensors alone or together to understand the duration or time and number of people in a given space, or if a room is occupied and how long it has been occupied, and CO₂ concentrations.

Based on an analysis of the above equation(s) as a function of real-time data in a space, the controller C may determine a risk level within that space. Using this calculated risk level, the controller C may cause the fan 10 to activate, deactivate, increase speed, decrease speed, and/or in some way adjust one or more of the associated germ-killing functions, such as by activating the source 16, in order to help deal with the presence of pathogens within the space, and thereby reduce the risk of disease transmission as a result.

A system including the fan 10 and controller C (which may be local or remote) may include software that utilizes the above equation(s) in a closed loop feedback system. As measured environmental parameters change, the system including the fan 10 may adjust the various elements of the system accordingly. A graphical user interface (GUI) may be provided for interaction with and/or control of the fan and/or other elements of the system. The GUI may be provided in association with a mobile application on a mobile device (e.g. a smart phone or a tablet), may be provided in association with a stationary or mobile remote control, and/or may be provided on the fan itself. In the case of a wall controller, the wall control may be bactericidal in nature.

In accordance with a further aspect of the disclosure, the fan blades 18 and/or any other element of the fan 10 may include a portion or coating (such as on a surface) that may act as a photocatalyst in an oxidation reaction for the purpose of destroying pathogens. As is known, the photocatalytic coating (such as, for example, titanium dioxide) may absorb light, such as from a UV source (lamp), and may form strong oxidizing hydroxyl radicals (—OH). To cause this reaction, the fan 10 may include a source 16 of germicidal energy, which may be in the form of a UV light for the purpose of activating the photocatalytic agent. These radicals formed in the presence of a UV light-absorbing photocatalytic agent may interact with the pathogens or other allergens or pollutants, thereby destroying the pathogens, allergens, or pollutants. As they decompose, the byproducts of the oxidation/decomposition are harmless and include water and carbon dioxide.

In the embodiment of FIG. 1, the source 16 is associated with the hub 12. In one aspect, the source 16 may be located above the fan blades 18, such that energy (e.g., light L as shown) projects outward across the top of the blades 18, but may comprise alternative arrangements as well, such as a UV-based uplight (see U.S. patent application Ser. No. 17/147,133, the disclosure of which is fully incorporated herein by reference, including as disclosing a fan with which the aspects of the present disclosure may be utilized). The light source 16 may be annular and surround the hub 12. In other embodiments, the source 16 may comprise multiple light sources around a perimeter of the hub 12, or instead of a light source, the source 16 may comprise a germicidal generator, such as an ion generator, associated with the fan (see U.S. patent application Ser. No. 17/147,086, the disclosure of which is fully incorporated herein by reference, including as disclosing a fan with which the aspects of the present disclosure may be utilized) or otherwise in communication with the controller C.

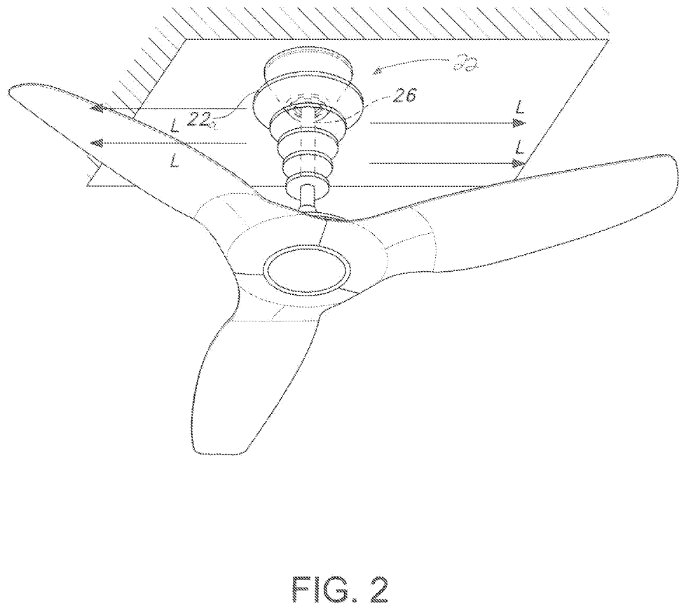

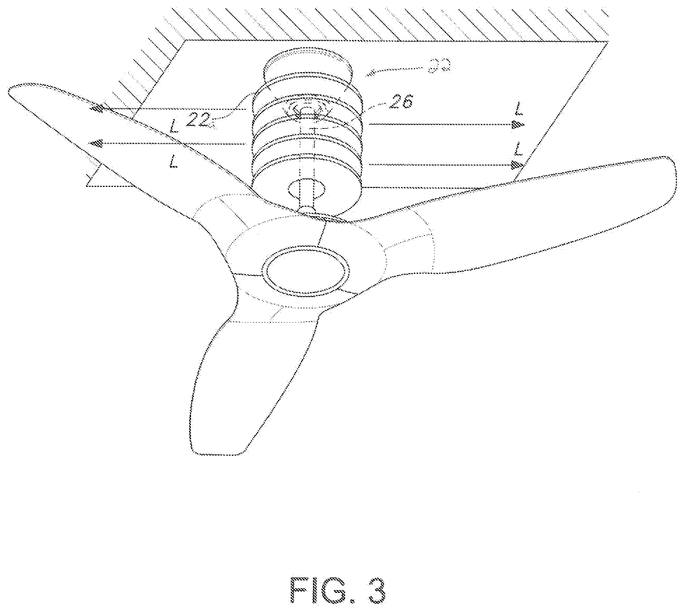

Turning now to FIGS. 2-3, the fan 10 in certain embodiments may include one or more reflectors 22 for reflecting and/or directing light L from an internal light source 26 onto the photocatalytic surfaces of the fan. The reflector(s) 22 may comprise a plurality of plates 22a, which may be substantially parallel with one another. Each plate 22a may have a reflective surface for reflecting light to a surface of the same plate or another plate including the photocatalyst (such that one plate may reflect light onto the photocatalytic surface of an adjacent plate, for example, or to its own photocatalytic surface).

In one aspect, the plates 22a may be substantially parallel with the floor or ground beneath the fan. In a further aspect, the plates may be closer together as they approach the hub 12, and may be farther apart as they are more distant from the hub 12. The shape of the plates 22 may be generally circular, or may take other shapes as desired.

The plates 22 may include reflective surfaces on the top and/or bottom thereof. When plates 22 are positioned adjacent or around the light source 26, as illustrated in FIGS. 2-3, the light L projected outwardly from the light source 26 may be reflected back and forth between adjacent plates 22. In this manner, the light L is directed in a direction generally parallel with the floor or ground beneath the fan 10. The plates 22 themselves, however, substantially prevent light L from travelling in a direction transverse to the plane of the parallel plates 22. This prevents light L, which may be in the form of UV light, directly onto a person in the space below the light.

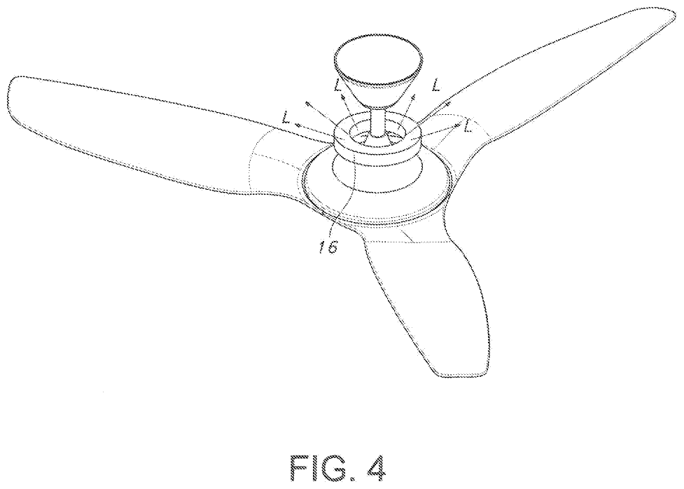

As can be seen in FIG. 2, the plates 22a may increase in size as they progress from the hub 12 toward the ceiling. In this manner, a tapered configuration may be provided. In another aspect, as illustrated in FIG. 4, the plates 22a may be generally similar in size. The plates 22a may extend an entire distance from the hub 12 to the ceiling, or may extend only along a portion of the distance.

With reference to FIG. 4, the light source 16 may generally form an annulus or donut shape. The light source 16 may be configured to direct light L upward and outward, thereby avoiding directing light at an occupant of the space below. The light source 16 may include one or more walls or other physical barriers for reflecting and/or directing the light L away from the space below.

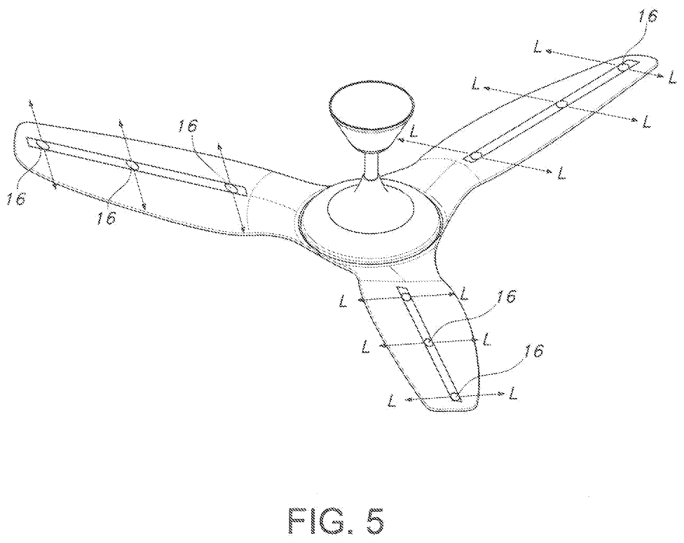

As illustrated in FIG. 5, the fan 10 may include a plurality of sources 16 of germicidal energy, such as lights. These sources 16 may be located along one or more surfaces of the blades 18. For instance, the fan may include a source 16 along a top surface of one or more blades 18. In another aspect, the fan 10 may include a light source 16 at an end of one or more of the blades 18. These sources 16 may be adapted to project energy, such as light L, in a direction other than downward into the space below the fan, such as for example toward the associated ceiling.

The fan 10 can be used to actively reduce the number of active airborne pathogens in a space or can trigger ventilation (such as from an external source (e.g., using a blower for causing an air exchange with the space) when more fresh air is needed. This can be implemented into ceiling fans or other electric fans of all sizes, from high-volume-low-speed fans a large as 24 feet in diameter to smaller residential fans that are four feet in diameter. Applicable space types include schools, offices, hospitals, fitness centers, public spaces/shopping malls, and homes.

Furthermore, automating the controls and having access to real-time and trended data may be used to implement risk management strategies and can save energy. For example, real time data can be collected and transmitted via local connections through technologies such as WiFi, Bluetooth, etc., including without internet connectivity. General data storage and analytics such as risk level trends, risk level alerts, equipment usage rates, as well as remote monitoring of real time data can occur via cloud connections locally or remotely from the fan.

Having shown and described various embodiments, further adaptations of the apparatuses, methods and systems described herein may be accomplished by appropriate modifications by one of ordinary skill in the art without departing from the scope of the disclosure. Several of such potential modifications have been mentioned, and others will be apparent to those skilled in the art. For instance, the examples, embodiments, geometries, materials, dimensions, ratios, steps, and the like discussed above are illustrative and are not required. Furthermore, while a ceiling fan 10 is shown, it should be appreciated that the disclosure could be applied to other fans, such as blowers, ventilators, HVAC units, exhaust fans, or the like, separately or in addition to a ceiling fan. Furthermore, while the sources 16, 26 are shown integral with the fan 10, it should be appreciated that they may be provided separate therefrom. The aspects of this disclosure may be applied to any fan of any document incorporated herein by reference, without limitation. Accordingly, the scope of the disclosure should be considered in terms of claims that may be presented, and is understood not to be limited to the details of structure and operation shown and described in the specification and drawings.

1. An apparatus for sterilizing and circulating air in a space, comprising:
a fan;
a source of germicidal energy;
a sensor for sensing a parameter of the space; and
a controller for controlling the fan or the source based on the sensed parameter.

2. The apparatus of claim 1, wherein the source comprises one or more UV lights attached to the fan.

3. The apparatus of claim 1, further comprising a reflector for reflecting light from the source onto a photocatalyst.

4. The apparatus of claim 3, wherein the reflector substantially surrounds the light source.

5. The apparatus of claim 3, wherein the reflector comprises a plurality of plates, each having a reflective surface.

6. The apparatus of claim 5, wherein the plurality of plates are substantially parallel to one another.

7. The apparatus of claim 5, wherein the plurality of plates are substantially parallel to a moveable part of the fan.

8. The apparatus of claim 1, wherein the controller is adapted to control the source based on the sensed parameter.

9. A system for minimizing exposure of at least one occupant to a pathogen and for circulating air within a space, comprising:
a fan;
at least one sensor for sensing a parameter within the space; and
a controller configured for determining a risk of exposure to the pathogen based on the sensed parameter within the space and to alter operation of the fan based on the determined risk of exposure.

10. The system of claim 9, wherein the at least one sensor comprises at least one of a temperature sensor, a humidity sensor, an occupancy sensor, and a carbon dioxide sensor.

11. The system of claim 9, wherein the controller is configured to alter a speed of the fan in response to the determined risk of exposure.

12. The system of claim 9, the fan includes a generator of germicidal energy.

13. The system of claim 12, wherein the generator comprises a UV light, and the controller is configured to activate the light based on the determined risk of exposure.

14. The system of claim 9, wherein the determined risk of exposure is based at least partially on a measured level of carbon dioxide in the space.

15. A method for circulating air within a space, comprising:
controlling a fan for circulating air within the space based on a determined risk of exposure to a pathogen.

16. The method of claim 15, further including the step of:
sensing a parameter within the space; and
determining the risk of exposure.

17. The method of claim 15, wherein determining the risk of exposure comprises determining a CO2 concentration or CO2 generation rate within the space.

18. The method of claim 15, wherein determining the risk of exposure comprises determining a probability of exposure.

19. The method of claim 15, further including the step of controlling a source of germicidal energy based on a determined risk of exposure to a pathogen.

20. The method of claim 15, further including the step of controlling a source of UV light associated with the space based on a determined risk of exposure to a pathogen.
